(19) United States
(12) Patent Application Publication
(10) Pub. No.: US 2021/0326787 A1
(43) Pub. Date: Oct. 21, 2021

(54) METHODS AND SYSTEMS OF PREDICTIVE ASSESSMENT OF FLIGHT SAFETY AND REAL-TIME RISK MITIGATION

(71) Applicant: Health Solutions Research, Inc., Rockville, MD (US)

(72) Inventor: Ajay Kumar Gupta, Potomac, MD (US)

(21) Appl. No.: 17/364,677

(22) Filed: Jun. 30, 2021

Related U.S. Application Data
(63) Continuation-in-part of application No. 17/107,407, filed on Nov. 30, 2020.
Application No. 17/107,407 is a continuation-in-part of application No. 16/887,608, filed on May 29, 2020.
Application No. 16/887,608 is a continuation-in-part of application No. 16/429,550, filed on Jun. 3, 2019.
Application No. 16/429,550 is a continuation-in-part of application No. 16/126,537, filed on Sep. 10, 2018.
Application No. 16/126,537 is a continuation-in-part of application No. 16/024,387, filed on Jun. 29, 2018.

(51) Int. Cl. G06Q 10/06; G06N 5/04; G16H 50/30; G16H 50/80; G16H 50/20; G06Q 10/02; B64D 45/00

(52) U.S. Cl. CPC G06Q 10/0635; G06N 5/04; G16H 50/30; G16H 50/80; G06Q 10/06375; G06Q 10/02; G06Q 10/06312; G06Q 10/06313; B64D 45/00; G16H 50/20

(57) ABSTRACT

A method of predictive assessment of flight safety and real-time risk mitigation is provided. The method includes determining a layout of an aircraft for a flight, generating a grid based on the determined layout of the aircraft, determining a list of passengers of the flight, and determining a transmission risk of a disease for each of the passengers of the flight. The method also includes predictively assessing a flight safety risk of the aircraft for the flight. The method further includes when the flight safety risk exceeds a predetermined threshold, rearranging the passengers to mitigate the flight safety risk, and adjusting the flight safety risk based on the rearranged passengers.

### CROSS-REFERENCE TO RELATED APPLICATIONS

This application is a continuation-in-part of U.S. application Ser. No. 17/107,407, filed on Nov. 30, 2020, which is a continuation-in-part of U.S. application Ser. No. 16/887,608, filed on May 29, 2020, which is a continuation-in-part of U.S. application Ser. No. 16/429,550, filed on Jun. 3, 2019, which is a continuation-in-part of U.S. application Ser. No. 16/126,537, filed on Sep. 10, 2018, which is a continuation-in-part of U.S. application Ser. No. 16/024,387, filed on Jun. 29, 2018, which claims benefit of provisional application 62/602,661, filed on May 1, 2017, the entire disclosures of which are hereby incorporated by reference herein.

### FIELD OF THE DISCLOSURE

The present disclosure is generally related to real-time risk assessment of disease transmission on a flight by predictively assessing risk reduction for airline operational decisions; and for assessing the risks associated with a particular aircraft used on a particular flight for analysis, intervention, and risk mitigation.

### BACKGROUND

A pandemic is an epidemic of infectious disease occurring on a scale that crosses international boundaries, affecting a substantial number of people. Throughout history, there have been a number of pandemics, including the Spanish flu of 1917, the Swine Flu of 1976, and COVID-19 (2019). COVID-19 is a respiratory virus that transmits from person to person via respiratory droplets found in aerosols that are produced when an infected person talks, coughs, sneezes, sings, or breathes. The virus has caused one of the worst public health crises in modern history.

Spreading over widespread geographic locations, pandemics typically affect a significant portion of the world-wide population, imposing its presence on every continent of the globe. A pandemic may affect virtually every aspect of everyday life, including home life, work life, and leisure time. As recent history shows, a novel virus may lead to, either progressively or simultaneously, international travel restrictions, the cancelation of numerous public events across the globe, and the quarantine and social isolation of millions and potentially billions of people. Prior to the pandemic of 2019-21, traveling to new places and exploring new cities and cultures, was one of the most beloved pastimes in our modern society, but during a deadly pandemic, has to consider the associated implications and potential risks with this once so popular endeavour.

There has been a growing body of research investigating and modelling the risk of viral transmission and the risk of death associated with the transmission of the virus. Looking forward, new approaches and modelling using the transmission risk index may be advantageous over the existing approaches.

### SUMMARY

Embodiments disclosed herein may provide real-time, predictive assessments of flight safety risk using a transmission risk index, passenger manifest, aircraft type, cabin layout, flight duration, air scrubbing duration, etc., to predict risks associated with particular aircraft(s) for particular flight(s) and to implement risk mitigation and preventive actions. Embodiments disclosed herein may pertain to the assessment of the value of health and safety mitigations in terms of disease transmission risk reduction on a per flight and per-aircraft basis, and further pertain to the assessment of the risk of disease transmission on each aircraft and each flight, thus allowing airlines or relevant decision makers (e.g., regulators, manufacturers, and passengers/travellers) to make predictive assessments of flight safety risk reduction on their operational decisions.

In at least one example embodiment, a method of predictive assessment of flight safety and real-time risk mitigation includes determining a layout of an aircraft for a flight; generating a grid based on the determined layout of the aircraft; determining a list of passengers of the flight; and determining a transmission risk of a disease for each of the passengers of the flight. The method also includes predictively assessing a flight safety risk of the aircraft for the flight by determining a plane transmission risk based on the transmission risk for each of the passengers, determining a passenger density score based on the grid and the passengers, and determining the flight safety risk based on the plane transmission risk, the passenger density score, a duration of the flight, and a scrubbing duration. The method further includes rearranging the passengers to mitigate the flight safety risk when the flight safety risk exceeds a predetermined threshold, and adjusting the flight safety risk based on the rearranged passengers.

In at least one example embodiment, a non-transitory computer-readable medium has computer-readable instructions that, if executed by a computing device, cause the computing device to perform operations including the above methods and/or other methods disclosed herein.

In at least one example embodiment, a system of predictive assessment of flight safety and real-time risk mitigation includes an aircraft and a controller. The controller is configured to determine a layout of the aircraft for a flight, generate a grid based on the determined layout of the aircraft, determine a list of passengers of the flight, and determine a transmission risk of a disease for each of the passengers of the flight. The controller is further configured to predictively assess a flight safety risk of the aircraft for the flight by determining a plane transmission risk based on the transmission risk for each of the passengers, determining a passenger density score based on the grid and the passengers, and determining the flight safety risk based on the plane transmission risk, the passenger density score, a duration of the flight, and a scrubbing duration. When the flight safety risk exceeds a predetermined threshold, the controller is further configured to rearrange the passengers to mitigate the flight safety risk and adjust the flight safety risk based on the rearranged passengers. It will be appreciated that rearranging any one or more passengers within the plane cabin based on their respective transmission risks and/or the flight safety risk can be performed individually (e.g., moving an individual passenger) or can be performed by incorporating groups of travellers (e.g., a family, a team, etc.) flying together.

### BRIEF DESCRIPTION OF THE DRAWINGS

The accompanying drawings illustrate various embodiments of systems, methods, and embodiments of various other aspects of the disclosure. Any person with ordinary skills in the art will appreciate that the illustrated element boundaries (e.g. boxes, groups of boxes, or other shapes) in the figures represent one example of the boundaries. It may be that in some examples one element may be designed as multiple elements or that multiple elements may be designed as one element. In some examples, an element shown as an internal component of one element may be implemented as an external component in another, and vice versa. Furthermore, elements may not be drawn to scale. Non-limiting and non-exhaustive descriptions are described with reference to the following drawings. The components in the figures are not necessarily to scale, emphasis instead being placed upon illustrating principles.

The present disclosure provides a detailed and specific description that refers to the accompanying drawings. The drawings and specific descriptions of the drawings, as well as any specific or alternative embodiments discussed, are intended to be read in conjunction with the entirety of this disclosure. The flight safety risk assessment and mitigation may, however, be embodied in many different forms and should not be construed as being limited to the embodiments set forth herein; rather, these embodiments are provided by way of illustration only and so that this disclosure will be thorough, complete and fully convey understanding to those skilled in the art.

References are made to the accompanying drawings that form a part of this disclosure and which illustrate embodiments in which the systems and methods described in this specification may be practiced.

FIG. 1 illustrates an example map showing a flight from a source location to a destination location, according to at least one example embodiment described herein.

FIG. 2 is a schematic diagram of factors in predicting or assessing a flight safety risk, according to at least one example embodiment described herein.

FIG. 3 shows a relationship between the passenger density score and the number of passengers, according to at least one example embodiment described herein.

FIG. 4 shows a relationship between the flight safety index and the passenger density score, according to at least one example embodiment described herein.

FIG. 5 shows a relationship between the flight safety index and the number of passengers, according to at least one example embodiment described herein.

FIG. 6 shows a relationship between the flight safety index and the transmission risk index, according to at least one example embodiment described herein.

FIG. 7 shows a relationship between the flight safety index and the duration of the flight, according to at least one example embodiment described herein.

FIG. 8 shows a relationship between the flight safety index and the number of passengers, according to at least one example embodiment described herein.

FIG. 9 shows a simulated relationship between the flight safety index and the number of passengers, according to at least one example embodiment described herein.

FIG. 10 shows simulated manifest distributions between the flight safety index and the likeliness of occurrence, according to at least one example embodiment described herein.

FIG. 11 shows a seat layout of an aircraft, according to at least one example embodiment described herein.

FIG. 12 is a schematic diagram of a seat layout of an aircraft, according to at least one example embodiment described herein.

FIG. 13 is a schematic diagram of a seat layout of an aircraft, according to at least another example embodiment described herein.

FIG. 14 is a schematic diagram of a seat layout of an aircraft, according to at least yet another example embodiment described herein.

FIG. 15 illustrates an example work flow of a method of predictive assessment of flight safety and real-time risk mitigation, according to at least one example embodiment described herein.

FIG. 16 illustrates an example grid of an aircraft, according to at least one example embodiment described herein.

### DETAILED DESCRIPTION

Although any systems and methods similar or equivalent to those described herein may be used in the practice or testing of embodiments of the present disclosure, the preferred, systems and methods are now described.

It will be appreciated that the operations, actions, steps, methods, instructions, and/or functions described herein may be stored in e.g., the computer program product and may be performed by e.g., the processor and/or the computing device of the processing system using e.g., the Transmission Risk Index (TRI) and the data source as described in U.S. application Ser. No. 17/107,407, the entire disclosure of which is hereby incorporated by reference herein.

As referenced herein, the terms “flight safety risk” or “flight safety index” may refer to a predictive assessment of a risk of a disease transmission on a particular aircraft and/or a particular flight. It will be appreciated that “disease” may be any contagious disease such as a viral, bacterial, or fungal disease. In at least one example embodiment, the virus may be a COVID variant or the like. In at least one example embodiment, the flight safety index for a particular flight may be determined based on the Transmission Risk Index (TRI), the passenger manifest, the aircraft type allocated to the flight, the cabin and seat layout of the aircraft, the projected flight duration, the projected aircraft scrubbing duration, etc. In at least one example embodiment, the flight safety index may be utilized to identify a risk of disease transmission on each flight to mitigate that risk and to restore passenger confidence in air travel. In at least one example embodiment, the higher the flight safety index is (or the lower the flight safety risk is), the safer the flight may be considered. In at least one example embodiment, the flight safety index may be the reciprocal of the flight safety risk.

As referenced herein, the terms “real-time” or “real time” may refer to various operations in computing or other processes for which response times may be a relatively short time, e.g., fractions of a second. A real-time process may generally be one that happens in defined time steps of maximum duration and fast enough to affect the environment in which it occurs, such as inputs to a computing system.

FIG. 1 illustrates an example map 100 showing a flight 110 from a source location to a destination location, according to at least one example embodiment described herein. The flight 110 includes an aircraft 120 assigned or allocated to the flight 110, and a flight schedule (not shown) including a duration of the flight, a scrubbing duration, a flight manifest, etc. It will be appreciated that unless explicitly specified otherwise, the flight described herein may refer to a scheduled flight that is yet to operate. That is, the duration of the flight may refer to the projected or estimated duration of the flight based on the flight schedule. The scrubbing duration may refer to the projected or estimated scrubbing duration based on the model and/or make of the aircraft per the flight schedule.

In at least one example embodiment, the duration of the flight, measured in minutes or hours, refers to the expected length of time for the aircraft to travel from the source location to the destination location. In at least one example embodiment, the duration of the flight may refer to the gate-to-gate travel time. In at least one example embodiment, the duration of the flight is measured in minutes.

In at least one example embodiment, the scrubbing duration, measured in minutes or hours, refers to the projected amount of time in which the volume of the internal space of the aircraft is circulated and thus replaced by fresh air (or the projected amount of time in which the air in the internal space of the aircraft is completely refreshed). In the example embodiments described herein, the scrubbing duration may alternatively be referred to as the air scrubbing duration or the air filtration duration. In at least one example embodiment, the scrubbing duration is measured in minutes.

It will be appreciated that the longer the scrubbing duration, the less frequently the scrubbings may occur in a given period of time. For example, in a ten-minute period of time, if the scrubbing duration is two minutes, five scrubbings may occur in the ten-minute period of time. If the scrubbing duration is five minutes, two scrubbings may occur in the ten-minute period of time. Thus, typically a longer scrubbing duration (in other words, the scrubbing is less often) may be associated with an increased risk of disease or viral transmission. Similarly, typically a longer flight duration may be associated with an increased risk of disease or viral transmission. It will be appreciated that in at least one example embodiment, on flights with a flight safety risk that is lower than an established risk threshold level, the scrubbing rate (e.g., with lower scrubbing rate or fewer scrubs in a given period of time or a longer interval between the scrubs) for those flights may be configured and/or controlled in order to e.g., save cost of operation.

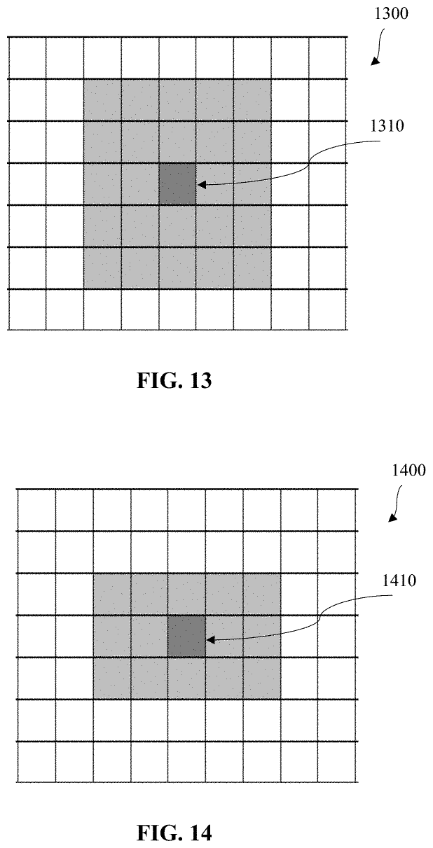

In at least one example embodiment, the duration of the flight and the scrubbing duration may be normalized by a close contact constant. “Close contacts,” as referenced herein, may refer to a number of instances of close contact with, e.g., someone who tested positive for a particular disease or virus within a particular amount of time. In at least one example embodiment, one instance of close contact with someone who tested positive for Covid-19 may be accounted for after at or around 15 minutes, and therefore the close contact constant may be 15. Thus, the duration of the flight and the scrubbing duration may be normalized with respect to the close contact constant, by, e.g., dividing each of the duration of the flight and the scrubbing duration by the close contact constant. It will be appreciated that the term “close contact neighbourhood” or “close contact radius” (described later with regard to FIG. 13) may have a different meaning from “close contact” or “close contact constant” as defined herein.

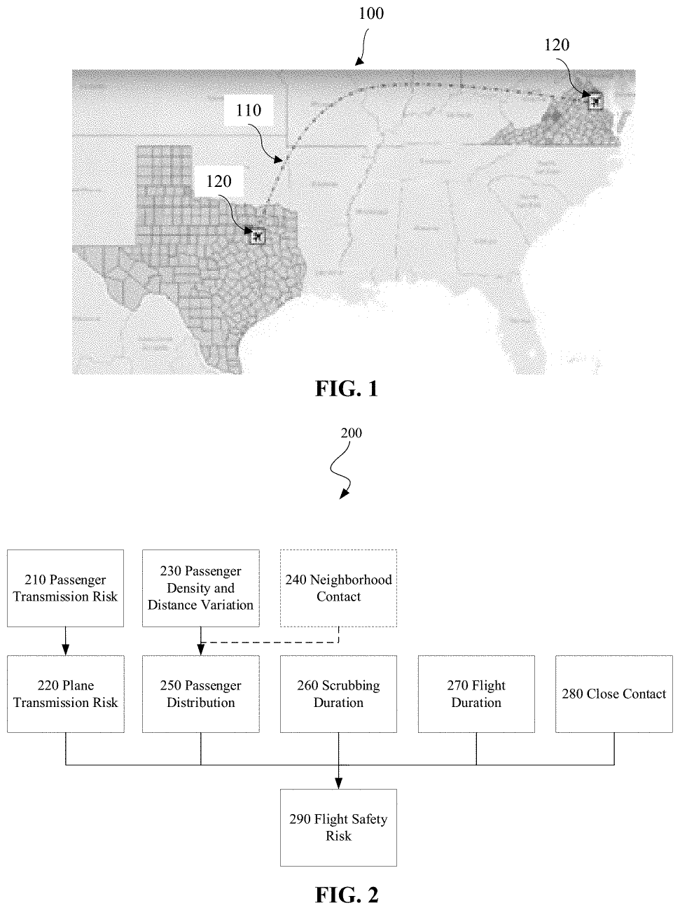

FIG. 2 is a schematic diagram 200 of factors considered for predicting or assessing a flight safety risk 290, according to at least one example embodiment described herein.

In at least one example embodiment, the Transmission Risk Index (TRI) may be utilized to identify a risk of disease transmission in a geographic area as well as assess environmental factors that may potentially exacerbate or inhibit the disease or viral transmission (e.g., incubation temperature for viral replication, etc.) and predict the spread and locations of future outbreaks at multiple geographic levels. In at least one example embodiment, the TRI may be a value between 0 and 100. In such embodiment, the TRI is dimensionless.

In at least one example embodiment, a manifest (e.g., a flight manifest or a passenger manifest) may include data regarding all the passengers booked to be on a particular flight. In at least one example embodiment, a manifest may include a list of passengers that are on the flight or to be on the flight, including, at least, zip codes thereof. The zip codes of the passengers may be used to determine the TRI associated with the geospatial locations. Thus, the TRI of the geospatial location for each particular passenger may be used to populate a grid of the aircraft with the associated TRI values. Each passenger arrives at the airport or terminal for a flight from a particular geographic area (e.g., at a Zip or postal code level) for which the TRI for that geographic area may be determined. The determined TRI of the particular geographic area may be assigned to or allocated to the passenger from that geographic area.

Block 210 (passenger transmission risk) represents the TRI for each passenger. The passenger transmission risk for a particular passenger is the TRI of the geographic area from which the particular passenger comes. The TRI of a geographic area is described in U.S. application Ser. No. 17/107,407, the entire disclosure of which is hereby incorporated by reference herein.

Block 220 (plane transmission risk) represents TRI for the aircraft, which may be an average TRI of all passengers on the aircraft. That is, the plane transmission risk may be an average of passenger transmission risk for all passengers on the aircraft. In at least one example embodiment, the passengers of the flight may include crew members.

Block 230 may represent the determined passenger density and the determined passenger distance variation (or inverse variation). An average of the passenger density and the passenger distance inverse variation may be referred to as passenger density score. In at least one example embodiment, a density of passengers may be determined by the number of passengers (e.g., booked on the flight) and the grid of the aircraft. A grid of an aircraft representing the layout of the aircraft may be generated based on the layout of the aircraft.

In at least one example embodiment, a preferred layout (e.g., a physical layout of the aircraft including e.g., a layout of seats within the cabin) of the aircraft allocated to the flight may be determined based on the make and/or model of the aircraft. In at least one example embodiment, the layout of the aircraft may be determined using e.g., machine learning process.

In at least one example embodiment, a grid of an aircraft may be referred to as a representation of an aircraft (e.g., interiors of the aircraft including cabin layouts, seats, aisles, etc.) for computational analysis by e.g., artificial intelligent module(s) using stochasticity (such as agent-based models and/or Monte Carlo simulation) to data-mine information related to flight safety index, including passenger distribution, passenger distance variation, and passenger density. It will be appreciated that in addition to or instead of a Monte Carlo simulation, other simulation measures may be employed. In at least one example embodiment, a grid of an aircraft may represent a capacity (e.g., a passenger capacity) and/or interior of a type of aircraft with seats layout including exit rows, aisles, class differences, potential extra space on the aircraft, etc. For example, a wide-body jet may have two aisles whereas a narrow-body jet may only have one, and the extra space resulting from the extra aisle may increase an average distance between all passengers.

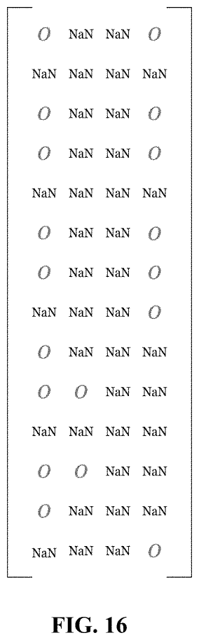

For example, FIG. 16 illustrates an example grid of an aircraft, according to at least one example embodiment described herein. As shown in FIG. 16, in the grid, “0” represents an occupy-able space such as an available seat for a passenger. “NaN” represents a non-occupy-able space that is not available for a passenger. When the “0” is occupied by a passenger (or booked by a passenger), the passenger manifest information (e.g., address of the passenger including the zip code and the TRI associated with the zip code, etc.) may be added into the grid for further analysis of the grid. The distance between passengers may be defined as the distance between the “O”s. An increase in distance between passengers may consequently result in a decrease of risk of disease transmission.

Referring back to Block 230 of FIG. 2, in at least one example embodiment, a density of passengers may be determined by the number of passengers (e.g., booked on the flight) and the grid of the aircraft. For example, the number of passengers booked on the flight may be divided by a total amount of space on the aircraft, which may be approximated by the grid of the aircraft for cabin area per volume, where the grid of the aircraft may take into consideration where the passengers may be located and have access to, excluding places such as the cockpit, or the like. In at least one example embodiment, the density of passengers may be an average density of the passengers on the aircraft. The density of passengers may represent the actual occupancy of the seats, the distances between the passengers on the flight, or the like. In the example embodiments described herein, “distance” or “distances” (e.g., between passengers, etc.) may be referred to as “distance” or “distances” in the context of the air flow patterns of the aircraft instead of a straight-line space between people on the aircraft. From a disease transmission perspective, the closeness of people on the aircraft is typically concerned with a possibility of one person's exposure to respiratory droplets (and/or other germ transmission) from another person. That is, the “distance” is based on the combination of a straight-line space (between people on the aircraft) and the air flow in the aircraft, since both factors may impact the movement and spread of respiratory droplets and/or germs.

In at least one example embodiment, a passenger distance variation or coefficient of variation for an average distance between the passengers on the aircraft may be determined, based on the number of passengers booked on the flight and the grid of the aircraft. The distance variation may be simulated or approximated via e.g., a Monte Carlo simulation. It will be appreciated that the simulation shows that there may be more variation in seat layout as the number of passengers actually on the aircraft decreases. It will also be appreciated that variation may be referred to as the ability for a passenger to change seats. For example, if the variation is high, a passenger may have more available seats to change to. If the variation is low, a passenger may have less available seats to change to. In at least one example embodiment, a passenger distance variation or coefficient of variation may be a dimensionless number (i.e., a number without a unit) that is less than one and equal to or greater than zero. The distance variation may be inversed to determine a passenger distance inverse variation, which may be desired to represent the risk of disease transmission since high variation may imply less flight safety risk since a passenger may have more available seats to change to, if the passenger chooses to be seated further away from other passengers instead of sitting close together with other passengers. In at least one example embodiment, the inverse of the distance variation may be one minus the value of the distance variation (which is a dimensionless number that is less than one and equal to or greater than zero), which may represent the extent to which the seat layouts of the passengers may be varied.

In at least one example embodiment, an average of the passenger density and the passenger distance inverse variation of Block 230 may be referred to as a passenger density score. FIG. 3 shows a relationship 300 between the passenger density score and the number of passengers, according to at least one example embodiment described herein. As shown in FIG. 3, the vertical coordinate represents the passenger density score for an aircraft and the horizontal coordinate represents the number of passengers in the aircraft. The passenger density score may be a function of the number of passengers on board the aircraft. For example, more passengers may imply more density. It will be appreciated that the maximum value of the passenger density score for a given number of passengers may be dependent on the types of the aircraft. As shown in FIG. 3, curves 310, 320, 330 represent different types of aircraft. In at least one example embodiment, the passenger density score is between 0 and 1. It will be appreciated that the passenger density score may be represented by any suitable value(s).

Referring back to Block 230 of FIG. 2, the passenger density score may be utilized as at least one example embodiment of the passenger distribution (Block 250). In at least another example embodiment, Block 250 may utilize Block 240 (neighbourhood contact, described later) instead of Block 230 as passenger distribution to determine the flight safety risk (Block 290).

Block 260 represents the scrubbing duration of the aircraft (see description of FIG. 1). Block 270 represents the duration of the flight (see description of FIG. 1). Block 280 represents the close contact constant (see description of FIG. 1).

In at least one example embodiment, the flight safety index may be determined or modelled as

$$100 \cdot [ 1 - {( 1 - \frac{DI}{100} )}^{1 + \frac{st}{{k}^{2}}} ] ,$$

where D represents the passenger distribution (Block 250), I represents the plane transmission risk (Block 220), s represents the scrubbing duration of the flight (Block 260), t represents the duration of the flight (Block 270), and k represents the close contact constant (Block 280). In at least one example embodiment, D may represent the passenger density score (Block 230). It will be appreciated that the interaction that time has with respect to the flight safety risk may not be necessarily linear. As such, the flight safety index model utilizes a probabilistic approach regarding the time such as the scrubbing duration and the duration of the flight. It will also be appreciated that in the flight safety index model, D*I are normalized (e.g., to be a value between 0 and 1) so that it may model the change in risk with respect to the number of times close contact has occurred in a probabilistic fashion. It will further be appreciated that the flight safety index model may at minimum, equal to the passenger density score times the plane TRI. It will also be appreciated that in at least one example embodiment, the flight safety index may be a value between 0 and 100, which may reflect that the TRI may also be between 0 and 100. In at least one example embodiment, the flight safety index may be the reciprocal of the flight safety risk (e.g., a value between 0% and 100% or the like). It will be appreciated that the flight safety risk/index may be represented by any suitable value(s).

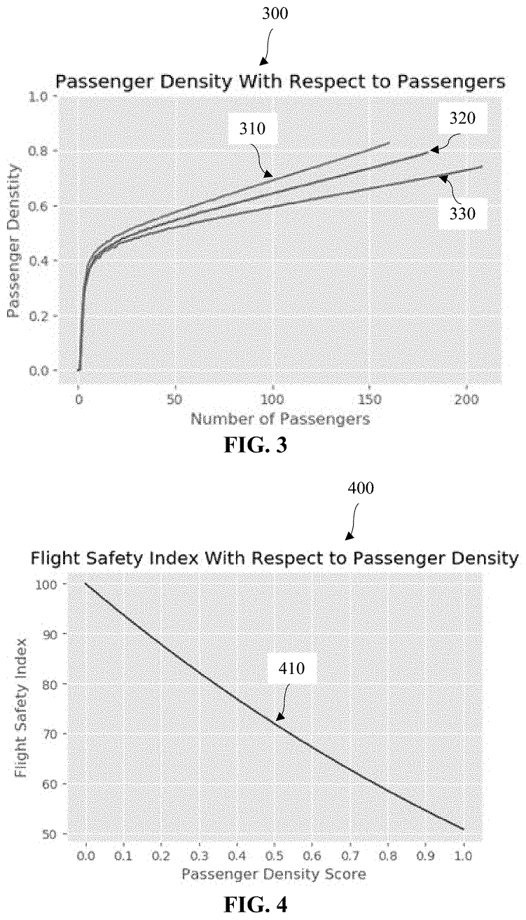

FIG. 4 shows a relationship 400 between the flight safety index and the passenger density score, according to at least one example embodiment described herein. As shown in FIG. 4, the horizontal coordinate represents the passenger density score for an aircraft assigned to a flight, and the vertical coordinate represents the flight safety index for the aircraft assigned to the flight. The flight safety index may be a function of the passenger density score. For example, reducing the passenger density score (i.e., reducing the flight density) may reduce the opportunity or risk for disease transmission (and thus a higher flight safety index). As shown in FIG. 4, the line 410 represents a relationship between the flight safety index and the passenger density score for a particular aircraft. In at least one example embodiment, the flight safety index is between 0 and 100. It will be appreciated that the flight safety index may be represented by any suitable value(s).

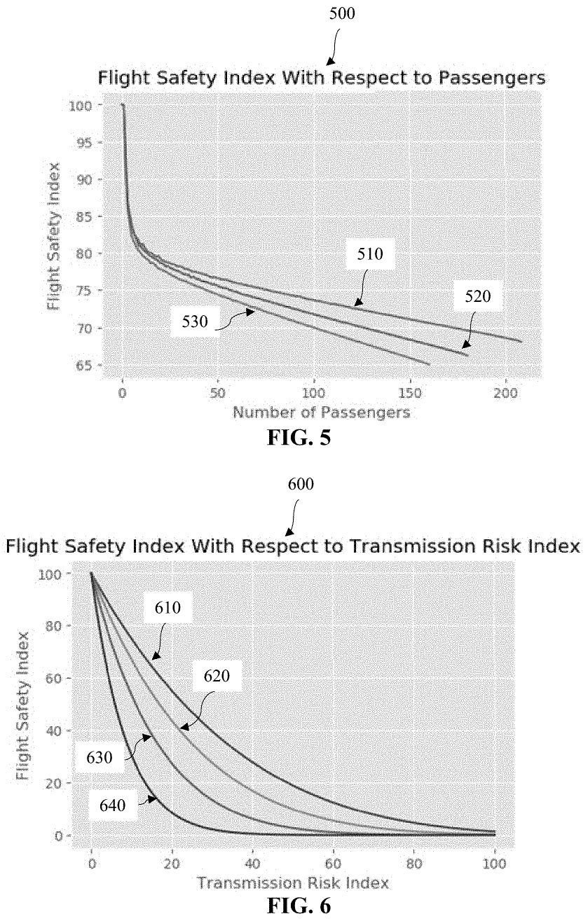

FIG. 5 shows a relationship 500 between the flight safety index and the number of passengers, according to at least one example embodiment described herein. As shown in FIG. 5, the horizontal coordinate represents the number of passengers for an aircraft assigned to a flight, and the vertical coordinate represents the flight safety index for the aircraft assigned to the flight. The flight safety index may be a function of the number of passengers. For example, more passengers may increase the risk of disease transmission (and thus a lower flight safety index). As shown in FIG. 5, curves 510, 520, 530 represent different types of aircraft. The aircraft 510 is larger than 520 which is larger than 530. The curves 510, 520, 530 indicate increased flight safety from switching to a larger aircraft.

FIG. 6 shows a relationship 600 between the flight safety index and the transmission risk index (e.g., plane transmission risk index or passenger transmission risk index), according to at least one example embodiment described herein. As shown in FIG. 6, the horizontal coordinate represents the transmission risk index for an aircraft assigned to a flight, and the vertical coordinate represents the flight safety index for the aircraft assigned to the flight. The flight safety index may be a function of the transmission risk index. For example, a larger transmission risk index may increase the risk of disease transmission (and thus a lower flight safety index). As shown in FIG. 6, curves 610, 620, 630, and 640 represent different air scrubbing duration. In at least one example embodiment, curve 610 represents a scrubbing duration of two minutes (in which the volume of the internal space of the aircraft is circulated in fresh air), curve 620 represents a scrubbing duration of three minutes, curve 630 represents a scrubbing duration of five minutes, and curve 640 represents a scrubbing duration of ten minutes. The curves 610, 620, 630, and 640 indicate decreased air scrubbing duration (i.e., increased air scrubbing frequency or rate in a given period of time) may reduce the opportunity for disease transmission (and thus a higher flight safety index for a given transmission risk index).

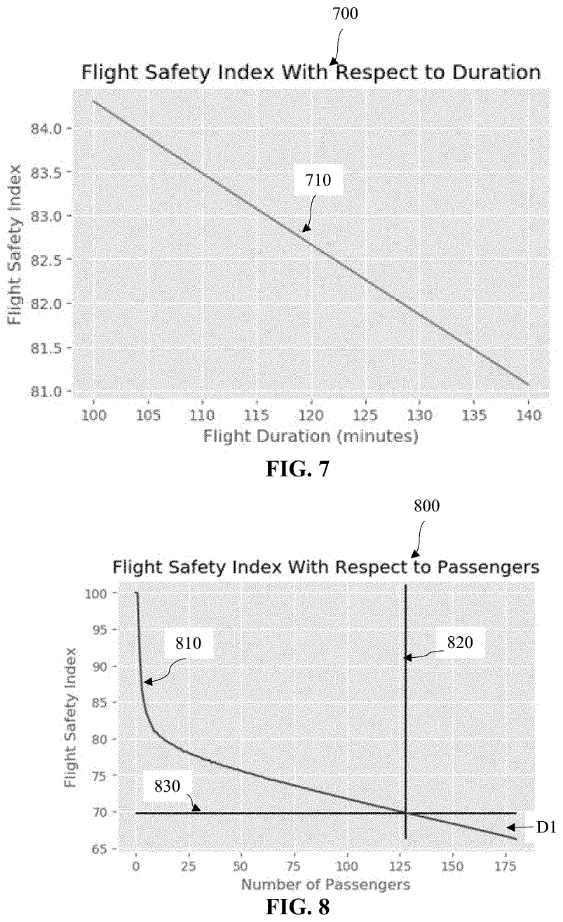

FIG. 7 shows a relationship 700 between the flight safety index and the duration of the flight, according to at least one example embodiment described herein. As shown in FIG. 7, the horizontal coordinate represents the flight duration, and the vertical coordinate represents the flight safety index for an aircraft assigned to the flight. The flight safety index may be a function of the flight duration. For example, reducing the flight duration may reduce the opportunity or risk for disease transmission (and thus a higher flight safety index). As shown in FIG. 7, the line 710 represents a relationship between the flight safety index and the flight duration.

FIG. 8 shows a relationship 800 between the flight safety index and the number of passengers, according to at least one example embodiment described herein. As shown in FIG. 8, the horizontal coordinate represents the number of passengers for an aircraft assigned to a flight, and the vertical coordinate represents the flight safety index for the aircraft assigned to the flight. The flight safety index may be a function of the number of passengers. For example, more passengers may increase the risk of disease transmission (and thus a lower flight safety index). As shown in FIG. 8, curve 810 represents a relationship between the flight safety index and the number of passengers without risk mitigation(s).

In at least one example embodiment, when the flight safety risk exceeds a predetermined threshold (or the flight safety index is below a predetermined threshold), risk mitigation actions may be taken to reduce the flight safety risk (or increase the flight safety index). One of the risk mitigation actions is blocking middle seats between passengers on the flight. As shown in FIG. 8, line 820 indicating a full flight with middle seats being blocked (e.g., 128 passengers for a particular aircraft, i.e., the maximum number of passengers that may fit in the particular aircraft when middle seats between passengers are blocked). Line 830 indicates the flight safety index value for a full flight with middle seats being blocked. FIG. 8 shows an assessment of a risk reduction from blocking the middle seats on a per flight basis. As shown in FIG. 8, D1 indicates an about 12% safety increase by blocking the middle seats for an aircraft having total 175 seats.

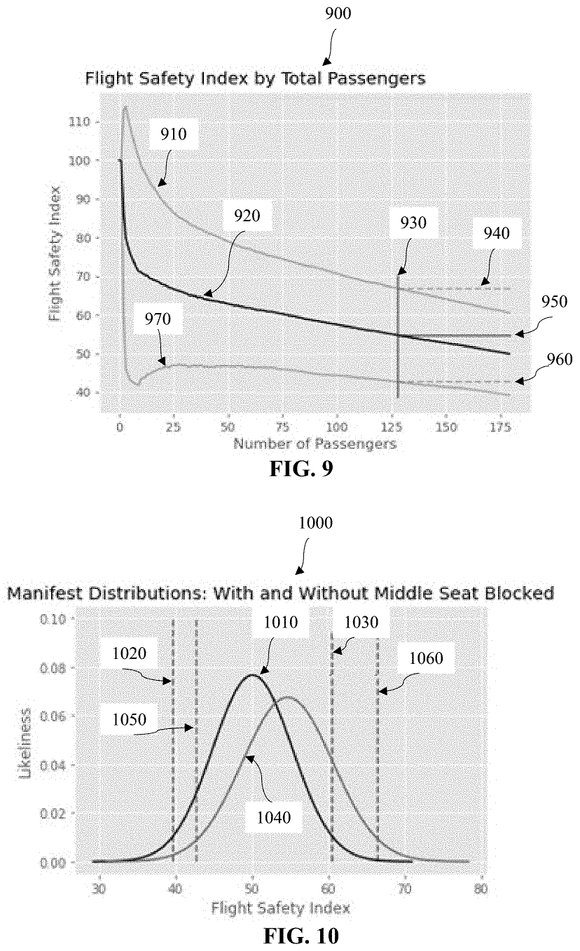

FIG. 9 shows a simulated relationship 900 between the flight safety index and the number of passengers, according to at least one example embodiment described herein. As shown in FIG. 9, the horizontal coordinate represents the number of passengers for an aircraft assigned to a flight, and the vertical coordinate represents the flight safety index for the aircraft assigned to the flight. The flight safety index may be a function of the number of passengers. For example, more passengers may increase the risk of disease transmission (and thus a lower flight safety index).

In at least one example embodiment, a model may be generated to simulate manifests (e.g., manifests including passengers from their respective metropolitan areas and/or their connecting airports) for a particular flight using a particular aircraft to test the impact of blocking the middle seat. For example, a passenger may be traveling to a destination location from a source location with a connecting airport/location. As shown in FIG. 9, curve 920 represents a simulated relationship or average trend between the flight safety index and the number of passengers, after sampling e.g., thousands of manifests.

As shown in FIG. 9, the difference in safety becomes larger when more passengers fill the aircraft beyond the capacity set by the middle seat blockage. The areas enclosed by lines 910 and 970 indicates at or about 95% of all sampled manifests giving an effective upper and lower bound to safety. The line 930 indicating a full flight with middle seats being blocked (e.g., 128 passengers for a particular aircraft, i.e., the maximum number of passengers that may fit in the particular aircraft when middle seats between passengers are blocked). The lines 910, 920, and 970 are extended to the right to make it easier for the comparison between a full flight with middle seats being blocked (940, 950, 960) and a full flight without seats blocking (910, 920, 970). For example, line 940 versus 910 to the right of line 930, line 950 versus 920 to the right of line 930, and line 960 versus 970 to the right of line 930 show such comparisons.

FIG. 10 shows simulated manifest distributions 1000 between the flight safety index and the likeliness of occurrence, according to at least one example embodiment described herein. It will be appreciated that the manifest distribution may be referred to as a distribution of the TRIs for the flight or passenger manifest. As shown in FIG. 10, the vertical coordinate represents the likeliness/possibility of occurrence in a distribution, and the horizontal coordinate represents the flight safety index for an aircraft assigned to a flight. Curve 1010 indicates manifest without middle seats being blocked. Curve 1040 indicates manifest with middle seats being blocked. Lines 1020 and 1030 indicate 95% of all sampled manifests for curve 1010, representing effective lower and upper bounds to safety. Lines 1050 and 1060 indicate 95% of all sampled manifests for curve 1040, representing effective lower and upper bounds to safety. FIG. 10 examines the comparison(s) of FIG. 9 further, with visualizing the exact differences, and the likelihood of all safety outcomes across all experimental trials. FIG. 10 shows a comparison between the distribution of flight safety index with and without middle seat blockage.

As shown in FIGS. 9 and 10, an aircraft cabin with middle seat blockage has a global increase in safety when compared to an aircraft without middle seat blockage. It will be appreciated that the benefit from middle seats blocking may vary depending on the passengers of the flight, or the plane transmission risk index. For some flights there may be a negligible benefit from blocking the middle seats with respect to the risk of disease transmission due to the inherent variation. It will also be appreciated that middle seats blocking may also have the benefits from passengers' comfort and/or perception of safety aboard the aircraft.

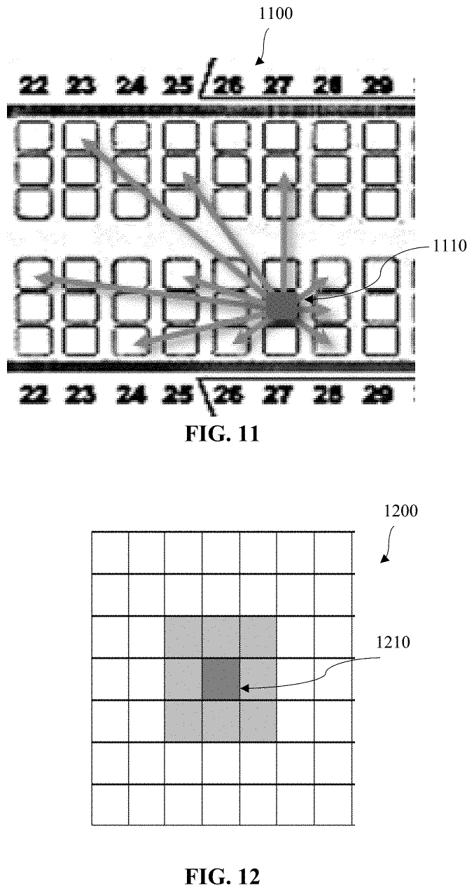

FIG. 11 shows a seat layout 1100 of an aircraft, according to at least one example embodiment described herein. As shown in FIG. 11, a passenger on the seat 1110 may have a neighbourhood or radius around the passenger on the aircraft.

FIG. 12 is a schematic diagram of a seat layout 1200 of an aircraft, according to at least one example embodiment described herein. As shown in FIG. 12, a passenger on the seat 1210 may have an adjacent contact neighbourhood or radius around the passenger on the aircraft.

FIG. 13 is a schematic diagram of a seat layout 1300 of an aircraft, according to at least another example embodiment described herein. As shown in FIG. 13, a passenger on the seat 1310 may have a close contact neighbourhood or radius around the passenger on the aircraft.

FIG. 14 is a schematic diagram of a seat layout 1400 of an aircraft, according to at least yet another example embodiment described herein. As shown in FIG. 14, a passenger on the seat 1410 may have a seatback contact neighbourhood or radius around the passenger on the aircraft.

As shown in FIGS. 11-14, each passenger on a seat may have a neighbourhood or radius around the passenger in the aircraft. Different types of neighbourhood or radius (e.g., adjacent contact neighbourhood, close contact neighbourhood, or seatback contact neighbourhood) may be a separate consideration that has separate assumptions with regard to flight safety risk. For example, in an aircraft or cabin with a compact seating arrangement, a close contact neighbourhood model may be applied since a distance between the passenger and other passengers within the close contact neighbourhood area may be less than or equal to a recommended social distance, and a distance between the passenger and other passengers outside the close contact neighbourhood area may exceed the recommended social distance.

In an aircraft or cabin with a loose seat arrangement, an adjacent contact neighbourhood model may be applied since a distance between the passenger and other passengers within the adjacent contact neighbourhood area may be less than or equal to the recommended social distance, and a distance between the passenger and other passengers outside the adjacent contact neighbourhood area may exceed the recommended social distance.

Also in an aircraft or cabin with a loose front and back seat arrangement, a seatback contact neighbourhood model may be applied since a distance between the passenger and other passengers within the seatback contact neighbourhood area may be less than or equal to the recommended social distance, and a distance between the passenger and other passengers outside the seatback contact neighbourhood area may exceed the recommended social distance. An average number of contacts in the specified radius may be determined for each of the seats in the aircraft (or for each passenger on the flight). It will be appreciated that during passenger check-in process, the neighbourhood or radius model may be particularly helpful because it allows for assessing a flight safety risk per subset of passengers on the flight (or per cabin, per sub-area of the internal space of the aircraft, etc.). It will also be appreciated that mitigation actions such as rearranging the seating arrangement for one or more passengers may be particularly useful with the neighbourhood or radius model.

Referring back to Block 240 of FIG. 2, Block 240 (neighbourhood contact) may represent the average number of contacts in the specified radius for each of the passengers/seats in the aircraft, and Block 250 (passenger distribution) may be determined based on Block 240 instead of Block 230 (passenger density and distance variation or distance inverse variation, or passenger density score). It will be appreciated that using Block 240 (neighbourhood contact) to determine Block 250 (passenger distribution) may be based on the assumption that that passengers may not rearrange seats to maximize the distance between themselves and others.

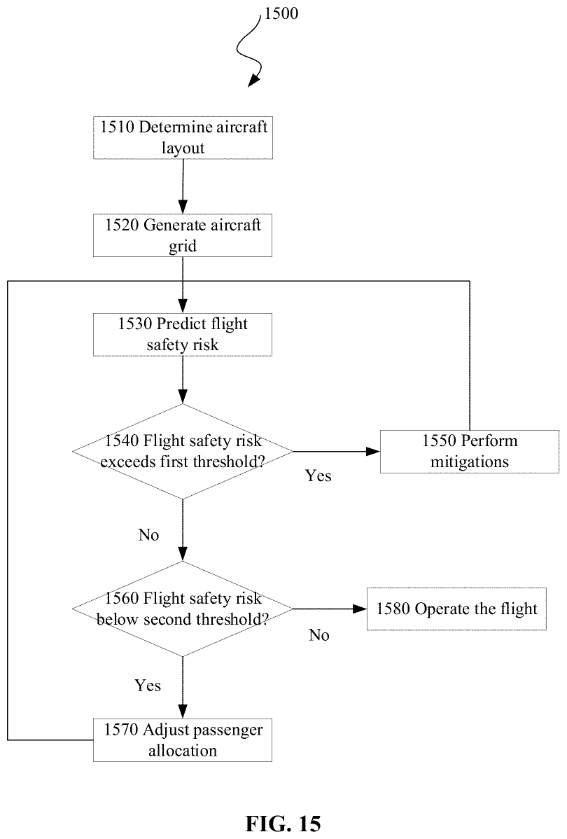

FIG. 15 illustrates an example work flow 1500 of a method of predictive assessment of flight safety and real-time risk mitigation, according to at least one example embodiment described herein. The work flow or processing flow 1500 may include one or more operations, actions, or functions depicted by one or more blocks 1510, 1520, 1530, 1540, 1550, 1560, 1570, and 1580. Although illustrated as discrete blocks, various blocks may be divided into additional blocks, combined into fewer blocks, or eliminated, depending on the desired implementation. As a non-limiting example, the description of processing flow 1500, corresponding to the depiction thereof in FIG. 15 and performed by a computing device, pertains to predictive assessment of flight safety, real-time monitoring the flight safety risk, real-time risk mitigation, and real-time reassessment of the flight safety risk after mitigation. Processing may begin at block 1510.

Block 1510 (Determine Aircraft Layout) may refer to the computing device determining a layout (e.g., a physical layout of the aircraft such as cabin and/or seats layout) of an aircraft assigned to a flight. In at least one example embodiment, the layout of the aircraft may be determined based on its make and model and layout data associated with the make and mode. The layout data includes the capacity (e.g., passenger capacity) of the aircraft, and the interior structure of the aircraft with seats layout including exit rows, aisles, class differences, potential extra space on the aircraft, airflow, or the like. In at least one example embodiment, the layout of the aircraft may be determined using e.g., machine learning process. In at least one example embodiment, the layout of the aircraft may be determined separately by class (e.g., economic class, business class, first class, or the like). Block 1510 may be followed by Block 1520.

Block 1520 (Generate Aircraft Grid) may refer to the computing device generating a grid representing the layout of the aircraft. In at least one example embodiment, the grid may digitally represent the capacity (e.g., passenger capacity) and/or interior structure of the aircraft. In at least one example embodiment, the grid may represent different classes of the seats layout. Block 1520 may be followed by Block 1530.

Block 1530 (Predict Flight Safety Risk) may refer to the computing device predicting or predictively assessing or determining the flight safety risk or flight safety index of the aircraft assigned to the flight. In at least one example embodiment, the predicting may be performed using the blocks, operations, actions, steps, methods, instructions, and/or functions described in FIGS. 2-7 and 11-14. Block 1530 may be followed by Block 1540.

Block 1540 (Flight Safety Risk Exceeds First Threshold) may refer to the computing device determining whether the predicted flight safety risk exceeds a first threshold (e.g., a predetermined threshold). In at least one example embodiment, Block 1540 may refer to the computing device determining whether the predicted flight safety index is below a threshold because the flight safety risk may be the reciprocal of the flight safety index. When the flight safety risk exceeds the first threshold (or the flight safety index is below a threshold), Block 1540 may be followed by Block 1550.

Block 1550 (Perform Mitigations) may refer to the computing device performing mitigation actions to reduce the flight safety risk (or to increase the flight safety index) so that the disease transmission risk on the aircraft for the flight may be reduced. In at least one example embodiment, Block 1550 may include blocking middle seats between the passengers on the aircraft for the flight using the blocks, operations, actions, steps, methods, instructions, and/or functions described in FIGS. 8-10.

In at least one example embodiment, Block 1550 may be performed in real-time in view of the real-time predicting and monitoring of the flight safety risk. For example, for an airline ticket reservation system, the flight safety risk may be predicted for the aircraft of the flight as soon as the system starts selling tickets (or accepting reservations of tickets). The flight safety risk may be zero when no ticket is sold/reserved since there may be no risk of disease transmission due to the fact that no passenger takes the flight. When a first person buys/reserves the ticket, there may be still no flight safety risk because there may be only one passenger on the flight and thus the passenger distribution or passenger density score may be zero. When a second person buys/reserves the ticket, there might be flight safety risk. As tickets are being sold by the system, which typically happens days (or weeks or months or the like) before the scheduled flight time, the system may track the level of flight safety risk in real-time and Block 1550 may be performed in real-time to reduce the flight safety risk. In at least one example embodiment, on the day of the scheduled flight time, during check-in process, the system may have another opportunity (e.g., by actively monitoring a check-in status of the passengers) to track the level of flight safety risk in real-time and Block 1550 may be performed in real-time to reduce the flight safety risk when people are checking in.

In at least one example embodiment, Block 1550 may include reducing the duration of the flight (e.g., by switching to a faster aircraft, or the like); decreasing air scrubbing duration (and thus increasing the air scrubbing frequency or rate in a given period of time); reducing the passenger distribution or passenger density score by e.g., cancelling a most recent incomplete action of reservation of an airline ticket for the flight, allocating a most recent complete reservation of an airline ticket for the flight to a second flight, shifting/moving passengers to other flight(s), switching to another aircraft having more passenger capacity, shifting/moving passengers to other classes in the same aircraft, adjusting seat allocations of the passengers within the class of the aircraft, or other mitigation actions as the airlines, regulators, or airports may deem appropriate to mitigate or reduce the risk of disease transmission on the flight.

In at least one example embodiment, Blocks 1510-1550 may be performed for one or more aircrafts assigned to one or more flights for one or more airlines. In such embodiment, the flight safety risks or flight safety indexes may be used for triggering aircraft retirement and/or procurement (e.g., when the flight safety risk(s) exceeds a threshold). Block 1550 may include retiring a first set of aircrafts and procuring a second set of aircrafts.

In at least one example embodiment, Blocks 1510-1550 may be performed for individual class (e.g., economic class, business class, first class, or the like) of an aircraft assigned to the flight instead of for the entire aircraft. Such embodiment may be achieved by separating the aircraft cabin and seats layouts by classes, and the flight safety risk may be predicted (and risk mitigations be performed) for each class instead of for the entire aircraft. It will be appreciated that such embodiment may provide a separate assessment of each core component of the aircraft cabin, and may influence the passenger densities greatly since first and business classes have less seats per unit area. It will also be appreciated that such embodiment may account for an airline's inability to move passengers between classes, thus being more relevant to their decision-making circumstances. By modelling both the whole aircraft's flight safety risk and the flight safety risk of separate class sections, more insight to the best actions airlines may take to mitigate risks on the flights may be provided.

In at least one example embodiment, Block 1550 may be followed by Block 1530 when the mitigation actions do not involve aircraft changes assigned to the flight. In at least one example embodiment, Block 1550 may be followed by Block 1510 when the mitigation actions involve aircraft changes assigned to the flight (e.g., by switching to bigger/faster/different aircraft(s)).

In at least one example embodiment, when the flight safety risk does not exceed the first threshold (or the flight safety index is not below a threshold), Block 1540 may be followed by Block 1560. It will be appreciated that in at least one example embodiment, Blocks 1560 and 1570 may be optional. In such embodiment, Block 1540 may be followed by Block 1580.

Block 1560 (Flight Safety Risk Below Second Threshold) may refer to the computing device determining whether the predicted flight safety risk is below a second threshold (e.g., a predetermined threshold). It will be appreciated that the airlines may establish as many thresholds as they deem appropriate. For example, the airlines may establish a particular threshold for each potential mitigation. That is, if threshold A is reached, mitigation one may be performed; if threshold B (that is greater than threshold A) is reached, mitigation two (alone or in addition to mitigation one) may be performed, etc. In at least one example embodiment, Block 1560 may refer to the computing device determining whether the predicted flight safety index is above another threshold because the flight safety risk may be the reciprocal of the flight safety index. When the flight safety risk is below the second threshold (or the flight safety index is above another threshold), Block 1560 may be followed by Block 1570.

Block 1570 (Adjust Passenger Allocation) may refer to the computing device adjusting passenger allocations to fully utilize the available spaces in the aircraft when the flight safety risk is in an acceptable range. For example, when there are a handful of passengers on an aircraft that may carry hundreds of passengers, the predicted flight safety risk may be well below a threshold (i.e., the second threshold, e.g., flight safety risk is at or about 5%) accepted by a user (e.g., the airline, etc.). In such case, passenger allocations may be adjusted to fully utilize the available spaces in the aircraft. In at least one example embodiment, Block 1570 may include cancelling the flight, and/or any adjustment that is opposite to or reverse of the risk mitigation actions of Block 1550. For example, Block 1570 may include increasing the passenger distribution or passenger density score by e.g., allocating reservation of an airline ticket from a second flight to this flight and shifting/moving passengers from other flight(s) to this flight, switching to another aircraft having less passenger capacity, increasing air scrubbing duration (and thus less expensive air ventilation equipment/device may be used or the energy consumed by air scrubbing may be reduced), or the like.

In at least one example embodiment, Block 1570 may be followed by Block 1530 when the adjustments do not involve aircraft changes assigned to the flight. In at least one example embodiment, Block 1570 may be followed by Block 1510 when the adjustments involve aircraft changes assigned to the flight (e.g., by switching to smaller/different aircraft(s)).

In at least one example embodiment, when the flight safety risk is not below the second threshold (or the flight safety index is not above another threshold), Block 1560 may be followed by Block 1580.

Block 1580 (Operate the Flight) may refer to the computing device or any other suitable operators/systems operating the flight.

It will be appreciated that embodiments of the flight safety risk/index disclosed herein may have advantageous to restore confidence in air travel, to develop cost-effective mitigation strategies, to minimize cost while maximizing risk reduction, to maximize sales while mitigating introduced risks, to determine how operational decisions may impact, and/or to lower overall risk of disease transmission via various mitigations including but not limited to constantly adding additional aircraft and routes, considering all operating seat layouts per aircraft, considering multiples aircraft and seat layout in use on each route, or the like. For example, simulation and testing show that reducing passenger density may have a significant reduction in the risk of disease transmission, swapping from a near full aircraft to a slightly larger aircraft may have a substantial positive impact on flight safety while changing a near empty aircraft to a smaller aircraft may have a small impact on flight safety.

Embodiments of the flight safety risk/index disclosed herein may assess the value of each mitigation effort in terms of disease transmission risk reduction, may be scalable to airline's full operating volume, and may deliver systematic results for reinforcing decisions that cut costs and saves lives. Embodiments of the flight safety risk model may allow the prediction of the flight safety risk without assumptions such as the knowledge of the infectious disease including the type of disease people already have, the level of infections, the level of viral load, or the like, which are typically available in, for example, a hospital environment. Embodiments of the flight safety risk/index disclosed herein may facilitate flexible and rapid implementation and/or integration into airline technical infrastructure, provide guidance on operational practices that minimize transmission risk and reduces airline operational costs, facilitate modelling of risk at the airport and throughout the travel experience, may serve as pilot program for route identification strategy, may involve multiple routes, may provide guidance on operational practices that minimize transmission risk and reduces airline operational costs, and may tailor flight safety to specific consumer needs. It will be appreciated that it may be determined (e.g., by user(s) such as an airline, regulators, etc.) that a particular flight route may have a flight safety risk that exceeds a predetermined threshold level, and a determination may be made to stop flights on that route temporarily or permanently as part of disease transmission risk mitigation.

Embodiments of the flight safety risk/index disclosed herein may be used to help reduce disease transmission and to evaluate the impact of disease transmission reduction they achieve. It will be appreciated that developing countermeasures may remain the role of the airlines, aviation industry groups, regulators, and/or airports. Embodiments of the flight safety risk/index disclosed herein may provide a system to measure risk with and/or without countermeasures so that the airlines, aviation industry groups, regulators, and/or airports may develop and select such.

One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

Different features, variations and multiple different embodiments have been shown and described with various details. What has been described in this application at times in terms of specific embodiments is done for illustrative purposes only and without the intent to limit or suggest that what has been conceived is only one particular embodiment or specific embodiments. It is to be understood that this disclosure is not limited to any single specific embodiments or enumerated variations. Many modifications, variations and other embodiments will come to mind of those skilled in the art, and which are intended to be and are in fact covered by both this disclosure. It is indeed intended that the scope of this disclosure should be determined by a proper legal interpretation and construction of the disclosure, including equivalents, as understood by those of skill in the art relying upon the complete disclosure present at the time of filing.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting.

### ASPECTS

It is understood that any one of aspects 1-20 may be combined with aspect 21.

Aspect 1. A method of predictive assessment of flight safety and real-time risk mitigation, the method comprising: determining a layout of an aircraft for a flight; generating a grid based on the determined layout of the aircraft; determining a transmission risk of a disease for each of the passengers of the flight; predictively assessing a flight safety risk of the aircraft for the flight by: determining a plane transmission risk based on the transmission risk for each of the passengers, determining a passenger density score based on the grid and the passengers, determining the flight safety risk based on the plane transmission risk, the passenger density score, a duration of the flight, and a scrubbing duration; when the flight safety risk exceeds a predetermined threshold, rearranging the passengers to mitigate the flight safety risk, and adjusting the flight safety risk based on the rearranged passengers.

Aspect 2. The method according to aspect 1, wherein determining the passenger density score includes: determining a density of the passengers based on the grid and the passengers, and simulating different distances between the passengers.

Aspect 3. The method according to aspect 2, wherein the simulating includes: performing a Monte Carlo simulation; and determining a distance inverse variation based on the Monte Carlo simulation.

Aspect 4. The method according to any one of aspects 1-3, wherein determining the passenger density score includes: determining a neighbourhood contact for each of the passengers of the flight based on the grid.

Aspect 5. The method according to aspect 4, wherein determining the neighbourhood contact for each of the passengers includes: determining an adjacent contact neighbourhood for each of the passengers.

Aspect 6. The method according to aspect 4, wherein determining the neighbourhood contact for each of the passengers includes: determining a close contact neighbourhood for each of the passengers.

Aspect 7. The method according to aspect 4, wherein determining the neighbourhood contact for each of the passengers includes: determining a seatback contact neighbourhood for each of the passengers.

Aspect 8. The method according to any one of aspects 1-7, further comprising: actively monitoring a check-in status of each of the passengers of the flight; and adjusting the flight safety risk based on the check-in status.

Aspect 9. The method according to aspect 8, further comprising: adjusting seat allocations of the passengers based on the adjusted flight safety risk; and reassessing the flight safety risk based on the adjusted seat allocations.

Aspect 10. The method according to aspect 8, further comprising: adjusting flight arrangements of the passengers based on the adjusted flight safety risk.

Aspect 11. The method according to aspect 10, wherein adjusting flight arrangements of the passengers based on the adjusted flight safety risk includes: when the adjusted flight safety risk exceeds the predetermined threshold, shifting a plurality of passengers to a second flight; and adjusting the flight safety risk based on the shifting of the plurality of passengers.

Aspect 12. The method according to aspect 10, wherein adjusting flight arrangements of the passengers based on the adjusted flight safety risk includes: when the adjusted flight safety risk below a second predetermined threshold, shifting a plurality of passengers from a second flight to the flight; and adjusting the flight safety risk based on the shifting of the plurality of passengers.

Aspect 13. The method according to any one of aspects 1-12, further comprising: predictively assessing flight safety risks for a plurality of flights; triggering aircraft retirement and/or procurement based on the flight safety risks.

Aspect 14. The method according to aspect 13, further comprising: retiring a first set of aircrafts and procuring a second set of aircrafts; adjusting the flight safety risks based on layouts of the second set of aircrafts.

Aspect 15. The method according to any one of aspects 1-14, wherein rearranging the passengers to mitigate the flight safety risk includes: allocating a most recent complete reservation of an airline ticket for the flight to a second flight.

Aspect 16. The method according to any one of aspects 1-15, wherein rearranging the passengers to mitigate the flight safety risk includes: cancelling a most recent incomplete action of reservation of an airline ticket for the flight.

Aspect 17. The method according to any one of aspects 1-16, wherein rearranging the passengers to mitigate the flight safety risk includes: blocking middle seats between the passengers in the flight.

Aspect 18. The method according to any one of aspects 1-17, wherein rearranging the passengers to mitigate the flight safety risk includes: switching the aircraft to a second aircraft for the flight; determining a layout of the second aircraft for the flight; generating a second grid based on the determined layout of the second aircraft; and reassessing the flight safety risk based on the second grid.

Aspect 19. The method according to any one of aspects 1-18, wherein the disease is an infectious disease.

Aspect 20. A non-transitory computer-readable medium having computer-readable instructions that, if executed by a computing device, cause the computing device to perform operations comprising the method of any one of aspects 1-19.

Aspect 21. A system of predictive assessment of flight safety and real-time risk mitigation, the system comprising: an aircraft; and a controller, wherein the controller is configured to determine a layout of the aircraft for a flight; generate a grid based on the determined layout of the aircraft; determine a list of passengers of the flight; determine a transmission risk of a disease for each of the passengers of the flight; and predictively assess a flight safety risk of the aircraft for the flight by: determining a plane transmission risk based on the transmission risk for each of the passengers, determining a passenger density score based on the grid and the passenger, and determining the flight safety risk based on the plane transmission risk, the passenger density score, a duration of the flight, and a scrubbing duration; wherein when the flight safety risk exceeds a predetermined threshold, the controller is further configured to rearrange the passengers to mitigate the flight safety risk and adjust the flight safety risk based on the rearranged passengers.

The examples disclosed in this application are to be considered in all respects as illustrative and not limitative. The terminology used in this specification is intended to describe particular embodiments and is not intended to be limiting. The terms “a,” “an,” and “the” include the plural forms as well, unless clearly indicated otherwise. The terms “comprising,” “having,” “containing,” and “including,” and other forms thereof, when used in this specification, specify the presence of the stated features, integers, steps, operations, elements, and/or components, but do not preclude the presence or addition of one or more other features, integers, steps, operations, elements, and/or components.

With regard to the preceding description, it is to be understood that changes may be made in detail, especially in matters of the construction materials employed and the shape, size, and arrangement of parts without departing from the scope of the present disclosure. This specification and the embodiments described are exemplary only, with the true scope and spirit of the disclosure being indicated by the claims that follow.

What is claimed is:

1. A method of predictive assessment of flight safety and real-time risk mitigation, the method comprising:
determining a layout of an aircraft for a flight;
generating a grid based on the determined layout of the aircraft;
determining a list of passengers of the flight;
determining a transmission risk of a disease for each of the passengers of the flight;
predictively assessing a flight safety risk of the aircraft for the flight by:
determining a plane transmission risk based on the transmission risk for each of the passengers,
determining a passenger density score based on the grid and the passengers,
determining the flight safety risk based on the plane transmission risk, the passenger density score, a duration of the flight, and a scrubbing duration;
when the flight safety risk exceeds a predetermined threshold, rearranging the passengers to mitigate the flight safety risk, and
adjusting the flight safety risk based on the rearranged passengers.

2. The method according to claim 1, wherein determining the passenger density score includes:
determining a density of the passengers based on the grid and the passengers, and
simulating different distances between the passengers.

3. The method according to claim 2, wherein the simulating includes:
performing a Monte Carlo simulation; and
determining a distance inverse variation based on the Monte Carlo simulation.

4. The method according to claim 1, wherein determining the passenger density score includes:
determining a neighbourhood contact for each of the passengers of the flight based on the grid.

5. The method according to claim 4, wherein determining the neighbourhood contact for each of the passengers includes:
determining an adjacent contact neighbourhood for each of the passengers.

6. The method according to claim 4, wherein determining the neighbourhood contact for each of the passengers includes:
determining a close contact neighbourhood for each of the passengers.

7. The method according to claim 4, wherein determining the neighbourhood contact for each of the passengers includes:
determining a seatback contact neighbourhood for each of the passengers.

8. The method according to claim 1, further comprising:
actively monitoring a check-in status of each of the passengers of the flight; and
adjusting the flight safety risk based on the check-in status.

9. The method according to claim 8, further comprising:
adjusting seat allocations of the passengers based on the adjusted flight safety risk; and
reassessing the flight safety risk based on the adjusted seat allocations.

10. The method according to claim 8, further comprising:
adjusting flight arrangements of the passengers based on the adjusted flight safety risk.

11. The method according to claim 10, wherein adjusting flight arrangements of the passengers based on the adjusted flight safety risk includes:
when the adjusted flight safety risk exceeds the predetermined threshold, shifting a plurality of passengers to a second flight; and
adjusting the flight safety risk based on the shifting of the plurality of passengers.

12. The method according to claim 10, wherein adjusting flight arrangements of the passengers based on the adjusted flight safety risk includes:
when the adjusted flight safety risk below a second predetermined threshold, shifting a plurality of passengers from a second flight to the flight; and
adjusting the flight safety risk based on the shifting of the plurality of passengers.

13. The method according to claim 1, further comprising:
predictively assessing flight safety risks for a plurality of flights;
triggering aircraft retirement and/or procurement based on the flight safety risks.

14. The method according to claim 13, further comprising:
retiring a first set of aircrafts and procuring a second set of aircrafts;
adjusting the flight safety risks based on layouts of the second set of aircrafts.

15. The method according to claim 1, wherein rearranging the passengers to mitigate the flight safety risk includes:
allocating a most recent complete reservation of an airline ticket for the flight to a second flight.

16. The method according to claim 1, wherein rearranging the passengers to mitigate the flight safety risk includes:
cancelling a most recent incomplete action of reservation of an airline ticket for the flight.

17. The method according to claim 1, wherein rearranging the passengers to mitigate the flight safety risk includes:
blocking middle seats between the passengers in the flight.

18. The method according to claim 1, wherein rearranging the passengers to mitigate the flight safety risk includes:
switching the aircraft to a second aircraft for the flight;
determining a layout of the second aircraft for the flight;
generating a second grid based on the determined layout of the second aircraft; and
reassessing the flight safety risk based on the second grid.

19. The method according to claim 1, wherein the disease is an infectious disease.

20. A non-transitory computer-readable medium having computer-readable instructions that, if executed by a computing device, cause the computing device to perform operations comprising the method of claim 1.
